(19) United States
(12) Patent Application Publication
(10) Pub. No.: US 2018/0078214 A1
(43) Pub. Date: Mar. 22, 2018

(54) PREDICTION METHOD AND DIETARY REGIME

(71) Applicant: MARS, INCORPORATED, McLean, VA (US)

(72) Inventors: John Flanagan, Aimargues (FR); Marie Anne Hours, Aimargues (FR); Patrick Nguyen, Nantes (FR); Chantal Thorin, Nantes (FR); Lucie Leclerc, Nantes (FR)

(73) Assignee: MARS, INCORPORATED, McLean, VA (US)

(21) Appl. No.: 15/711,915

(22) Filed: Sep. 21, 2017

Related U.S. Application Data
(60) Provisional application No. 62/444,079, filed on Jan. 9, 2017.

(30) Foreign Application Priority Data
Sep. 21, 2016 (EP) 16306212.8

(51) Int. Cl. A61B 5/00; A23K 50/40; A23L 33/00; A61B 5/107; A01K 29/00

(52) U.S. Cl. CPC A61B 5/7275; A23K 50/40; A61B 2503/40; A61B 5/107; A01K 29/005; A23L 33/40

(57) ABSTRACT

The presently disclosed subject matter relates to methods of determining whether a canine animal is predisposed to being overweight in adulthood, and methods of preventing a canine animal from being overweight in adulthood. The present application also provides a diet or a dietary regime for use in preventing a canine animal from being overweight in adulthood.

### CROSS-REFERENCE TO RELATED APPLICATIONS

The present application claims priority to European Application No. 16306212.8, filed on Sep. 21, 2016, and to U.S. Provisional Application No. 62/444,079, filed on Jan. 9, 2017, the contents of which are both incorporated herein by reference in their entireties.

### FIELD

The presently disclosed subject matter relates to a prediction method and to a diet and/or dietary regime.

### BACKGROUND

Obesity and (overweight) is now a global concern for dogs, and its prevalence is increasing. Obesity is the most common medical disease in companion animals and is associated with comorbidities such as orthopaedic disease, arthrosis, cardiorespiratory diseases and diabetes mellitus, as well as causing metabolic derangements, altered renal function and other respiratory dysfunction. In addition to these adverse effects on health, quality of life is poorer in obese and overweight dogs and lifespans can be shortened.

Over-feeding and reduced metabolic activity are regularly provided as the main reasons behind the increasing prevalence of overweight and obesity. Nevertheless, excess energy intake and reduced metabolic activity do not fully explain why some dogs become obese and some do not. To date, risk prediction factors for development of obesity in dogs are not known.

Given the obesity-related issues and the operating system of the energy balance, there remains a need for a better strategy to prevent fat mass development than managing weight loss programs.

### SUMMARY OF THE INVENTION

The presently disclosed subject matter relates to methods of determining whether a canine animal is predisposed to being overweight in adulthood, and methods of preventing a canine animal from being overweight in adulthood. The present application also provides a diet or a dietary regime for use in preventing a canine animal from being overweight in adulthood.

In certain non-limiting embodiments, the presently disclosed subject matter provides a method of determining whether a canine animal is predisposed to being overweight in adulthood. In certain embodiments, the method comprises:

(i) determining a first weight of the canine animal at birth;

(ii) determining a second weight of the canine animal at a date between about 13 to about 15 days of age; and

(iii) determining a weight gain between the dates of steps (i) and (ii),

wherein a weight gain of about 125% or more between the dates of steps (i) and (ii) determines that the canine animal is predisposed to being overweight in adulthood.

In certain embodiments, the date of the second weight is about 14 days of age.

In certain embodiments, adulthood is from about 2 years of age onward.

In certain embodiments, overweight is obese.

In certain embodiments, the canine animal is a domestic dog.

In certain embodiments, wherein the canine animal is determined to be predisposed to be overweight in adulthood, a diet or a dietary regime is administered to maintain or achieve a healthy bodyweight.

In certain non-limiting embodiments, the presently disclosed subject matter provides a diet or a dietary regime for a canine animal, which has been determined as predisposed to being overweight in adulthood according to the method disclosed herein.

In certain embodiments, the dietary regime contains, on a dry matter basis,

(i) an energy density of less than about 4100 kcal/kg;

(ii) less than about 20% w/w or less than about 15% w/w fat;

(iii) less than about 25% w/w or less than about 20% w/w carbohydrate;

(iv) more than about 30% w/w protein; or

(v) more than about 5% w/w or more than about 10% w/w total dietary fiber.

In certain non-limiting embodiments, the presently disclosed subject matter provides a method of preventing a canine animal from being overweight in adulthood. In certain embodiments, the method comprises:

(i) determining a first weight of the canine animal at birth;

(ii) determining a second weight of the canine animal at a date between about 13 to about 15 days of age; and

(iii) determining a weight gain between the dates of steps (i) and (ii), wherein a weight gain of 125% or more between the dates of steps (i) and (ii) determines that the canine animal is predisposed to being overweight in adulthood, and

(iv) administering a maintenance diet for a maintenance period, or administering a weight adjustment diet for a weight adjustment period.

In certain embodiments, the weight adjustment diet is a weight loss diet or a weight gain diet.

In certain embodiments, the method further comprising determining a maintenance body weight of the canine animal, comparing the maintenance body weight to a healthy body weight to calculate a body weight deviation, and administering a weight adjustment diet if the body weight deviation is about 5% or greater.

In certain embodiments, the weight adjustment diet is a weight loss diet if the body weight deviation is above the healthy body weight.

In certain embodiments, the weight adjustment diet is a weight gain diet if the body weight deviation is below the healthy body weight.

In certain embodiments, administering a maintenance diet or administering a weight adjustment diet is replaced or supplemented by administering a therapeutic agent.

In certain embodiments, the therapeutic agent is capable of treating or preventing obesity.

In certain non-limiting embodiments, the presently disclosed subject matter provides a diet or a dietary regime for use in preventing a canine animal from being overweight in adulthood, wherein the diet or the dietary regime is administered after the method is carried out, wherein the canine animal has been determined as predisposed to being overweight in adulthood.

The foregoing has outlined rather broadly the features and technical advantages of the present application in order that the detailed description that follows may be better understood. Additional features and advantages of the application will be described hereinafter which form the subject of the claims of the application. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of the application, both as to its organization and method of operation, together with further objects and advantages will be better understood from the following description

### BRIEF DESCRIPTION OF THE DRAWINGS

The disclosed subject matter is described with reference to the Figures.

FIGS. 1A and 1B show outputs of the PCA followed by the hierarchical clustering on principal components (HCPC), assessed on 24 female Beagle dogs aged 24 months. Groups were defined as ideal weight (IW, n=9), slightly overweight (OW1, n=6) or severely overweight (n=9). FIG. 1A: Factor map of principal and supplementary variables. The principal variables (solid lines) are fat-free mass (FFM, kg), fat mass proportion (FM %, %) and pelvic circumference (PC, cm). FIG. 1B: Confidence ellipses (95% confidence level) around the groups identified by (HCPC).

FIG. 2 shows the number of dogs of each BCS at considered ages. Groups were defined as ideal weight (IW, n=9), slightly overweight (OW1, n=6) or severely overweight (OW2, n=9).

FIG. 3 shows the body weight as a function of time in 24 female Beagle dogs. Values represent means of groups, while error bars represent SEM. Groups were defined as ideal weight (IW, n=9), slightly overweight (OW1, n=6) or severely overweight (OW2, n=9). * significant difference (p<0.05) between groups as identified by a linear regression model.

FIG. 4 shows a scatter plot of GR_{2W} (%) of three groups, during the two first weeks of life. Groups were defined as ideal weight (IW, n=9), slightly overweight (OW1, n=6) or severely overweight (OW2, n=9). Data are presented by groups, the line represents the median of the group; error bars represent the interquartile range. * significant difference (p<0.05) between groups as identified by a linear regression model.

### DETAILED DESCRIPTION

The presently disclosed subject matter relates to methods of determining whether a canine animal is predisposed to being overweight in adulthood, and methods of preventing a canine animal from being overweight in adulthood. The present application also provides a diet or a dietary regime for use in preventing a canine animal from being overweight in adulthood. For clarity and not by way of limitation, the detailed description of the presently disclosed subject matter is divided into the following subsections:

1. Definitions;

2. Methods;

3. Diet;

4. Foodstuff; and

5. Device and System.

### 1. Definitions

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “adulthood” refers to the time in an animal's life when they are fully grown. Adulthood of a canine animal is typically defined as a time of from about two years of age onward. By about two years, all dogs should be fully grown. Dogs are often described with reference to three main size groups: small dogs are less than about 25 lbs when fully grown; medium/large dogs are about 25-60 lbs when fully grown; and giant dogs are usually over about 60 lbs when fully grown. Small dogs are typically fully grown by about 12 months. Medium/large dogs are typically fully grown by about 18 months. Giant dogs are typically fully grown by about 24 months.

The life stages of puppyhood and adolescence are often used with reference to dogs, in particular with reference to the foodstuffs provided to meet their nutritional and energy requirements. Puppyhood starts from birth and typically ends at about 6-18 months, depending on the animal size (small, medium/large, giant) as defined above. Adolescence starts after puppyhood ends, i.e., about 6-18 months, depending on the animal size. The terms “adolescent” and “junior” dog have the same meaning. The term ‘adolescent’ is used in this application.

As used herein, the term “normal weight” or “healthy” means when an animal's weight or body mass is within about two centiles of its projected growth curve.

As used herein, the term “unhealthy” means the animal is obese, overweight, or underweight.

As used herein, the term “overweight” includes “obese” and “obesity.” Overweight dogs carry excessive accumulation of adipose tissue of more than about 15% of total body weight. Obese dogs carry excessive accumulation of adipose tissue of more than about 30% of total body weight. Ideal body weight is defined as the weight of the dog with a body condition score (BCS) rating of 5 on a 9 point scale, as described in the journal article by Laflamme D, in Canine Practice, July/August 1997, vol 22, No. 4, pages 10-15, entitled “Development and Validation of a Body Condition Score System for Dogs”, incorporated herein by reference.

A canine animal is a member of biological family Canidae, which is a lineage of carnivorans that includes domestic dogs, wolves, foxes, jackals, dingoes, and many other dog-like mammals. In certain embodiments, a canine animal is a domestic dog. Domestic dog breeds which are included in the presently disclosed subject matter include, but are not limited to, Labrador Retriever, Golden Retriever, Beagle, Chihuahua, Jack Russell Terrier, Pug, Labrador, Yorkshire Terrier, Dachshund, Rottweiler, Border Collie, Cavalier King Charles Spaniel, Carlino, Boxer, Poodle, Shih Tzu, Shetland Sheepdog, Teckel, Cocker Spaniel, German Shepherd, Maltese, Schnauzer, Staffordshire Bull Terrier, West Highland White Terrier, Cocker Spaniel, English Bulldog, including any potential cross-breeds of breeds included or not in the aforementioned list.

The term “user” as used herein includes, for example, a person or entity that owns a computing device or wireless device; a person or entity that operates or utilizes a computing device or a wireless device; or a person or entity that is otherwise associated with a computing device or wireless device. It is contemplated that the term “user” is not intended to be limiting and may include various examples beyond those described. In certain embodiments, the disclosed subject matter can be used by dog breeders and dog owners.

### 2. Methods

In certain non-limiting embodiments, the presently disclosed subject matter provides for methods of determining that a canine animal is predisposed to being overweight in adulthood. In certain embodiments, the method comprises:

(i) determining a first weight of the canine animal at birth;

(ii) determining a second weight of the canine animal at a date between about 13 to about 15 days of age; and

(iii) determining a weight gain between the dates of steps (i) and (ii),

wherein a weight gain of about 125% or more between the dates of steps (i) and (ii) determines that the canine animal is predisposed to being overweight in adulthood.

In certain embodiments, the time period in (ii) is about 10 days of age, about 10.5 days of age, about 11 days of age, about 11.5 days of age, about 12 days of age, about 12.5 days of age, about 13 days of age, about 13.5 days of age, about 14 days of age, about 14.5 days of age, about 15 days of age, about 15.5 days of age, about 16 days of age, about 16.5 days of age, about 17 days of age, or any intermediate date or period thereof. In certain embodiments, the time period in (ii) is about 14 days of age.

In certain embodiments, a weight gain of about 125%, about 130%, about 135%, about 140%, about 145%, about 150%, about 160%, about 170%, about 180%, about 190%, about 200%, about 250%, about 300%, about 350%, about 400%, about 450% or more, or any intermediate value or range thereof between the dates of steps (i) and (ii) determines that the canine animal is predisposed to being overweight in adulthood.

In certain embodiments, the canine animal is a domestic dog. In certain embodiments, the domestic dog is a small dog, wherein the adulthood is from about 2 years of age onward or from about 1 year of age onward. In certain embodiments, the domestic dog is a median or large dog, wherein the adulthood is from about 2 years of age onward or from about 18 months of age onward. In certain embodiments, the domestic dog is a giant dog, wherein the adulthood is from about 2 years of age onward.

In certain embodiments, interventions can be implemented. In certain embodiments, a diet or a dietary regime is administered before and/or after adulthood is reached, preferably beforehand if a predisposition to being overweight is determined.

The steps of determining the weight of the canine animal at steps (i) and (ii) can be by any methods known in the art. In certain embodiments, the weight of the canine animal is determined by weighing the animal on standard weighing scales (electronic or other). In certain embodiments, measurement of body weight at birth is performed within the first 1, 2, 3, 4, 5, 6, 7, 8, 9, or 10 hours, or any intermediate value or range thereof after birth. In certain embodiments, measurement of body weight at birth is performed within the first 3 hours after birth. In certain embodiments, the body weight at birth is measured on a standard weighing scale capable of reading to +/−1 g. In certain embodiments, all other weighing is performed at the same phase of the day for each animal, for example, after or prior to a feed.

In certain embodiments, this disclosed subject matter solves the problem by the identification of elevated risk of overweight/obesity in dogs at a young age. Specifically, puppies which increased their body weight by more than about 125% in the first 2 weeks of life were found to be obese at about 2 years of age.

In certain embodiments, the method is used by dog breeders and dog owners to evaluate the risk of obesity of puppies from a very young age. In this way, the breeders and owners will be alerted to the elevated level of risk which pertains to these dogs and will be sensitive to any minor increases in their dog's body fat. By increasing the owner's awareness of their dog's overweight/obesity development risk, preventative measures (such as reduction in calorie allocation or increase in physical exercise) can be employed to eliminate the development of the effects of overweight/obesity.

In certain non-limiting embodiments, the presently disclosed subject matter provides a method of preventing an animal being overweight in adulthood. In certain embodiments, the method comprising:

(i) determining a first weight of the canine animal at birth;

(ii) determining a second weight of the canine animal at a date between about 13 to about 15 days of age; and

(iii) determining a weight gain between the dates of steps (i) and (ii), wherein a weight gain of 125% or more between the dates of steps (i) and (ii) determines that the canine animal is predisposed to being overweight in adulthood, and

(iv) administering a maintenance diet for a maintenance period, or administering a weight adjustment diet for a weight adjustment period.

In certain embodiments, where the animal is determined to be predisposed to being overweight in adulthood, the weight adjustment diet can be a weight loss diet. In other embodiments, the weight adjustment diet is a weight gain diet.

In certain embodiments, the method further comprises determining a maintenance body weight of the canine animal, comparing the maintenance body weight to a healthy body weight to calculate a body weight deviation, and administering a weight adjustment diet if the body weight deviation is about 5% or greater. In certain embodiments, the body weight deviation is about 6%, about 7%, about 8%, about 9%, about 10%, about 15%, about 20%, about 25%, about 30% or greater, or any intermediate value or range thereof. In certain embodiments, the maintenance body weight is a body weight determined when the animal enters adulthood or any time afterwards. In certain embodiments, the maintenance body weight can be determined by any method, including weighing the animal on standard weighing scales (electronic or other).

In certain embodiments, the weight adjustment diet is a weight loss diet if the body weight deviation is above the healthy body weight. In certain embodiments, the weight adjustment diet is a weight gain diet if the body weight deviation is below the healthy body weight.

In certain embodiments, administering a maintenance diet or administering a weight adjustment diet is replaced or supplemented by administering a therapeutic agent, preferably a therapeutic agent to prevent the canine animal from being overweight in adulthood and maintaining good health. In certain embodiments, the therapeutic agent is capable of treating or preventing obesity.

### 3. Diet

The presently disclosed subject matter further provides diets, a dietary regime and/or behavioural intervention for a canine animal which has been determined as predisposed to being overweight in adulthood according to a method disclosed herein. According to the methods disclosed herein, the animal which has been determined to be predisposed to be overweight in adulthood can have a diet, a dietary regime and/or behavioural intervention put in place to maintain or achieve a healthy bodyweight of the animal. In certain embodiments, the diet and/or dietary regime is low in fat, low in energy, provide satiating effects to the animal, can reduce anxiety and stress, can reduce signs of begging, can enhance quality of life and/or can enhance activity levels of the animal. In certain embodiments, the behavioural intervention can include increasing the activity/exercise of the animal.

The diet and/or dietary regime can be any diet and/or dietary regime, including those which are already known in the art, and those to be developed. In certain embodiments, the diet and/or dietary regime is preferably started or used as soon as the determination of the first aspect is made. In certain embodiments, “as soon as” means immediately (the next meal) or within about 1, 2, 3 or 4 weeks, although any time period is contemplated. In certain embodiments, “as soon as” includes waiting for any growth spurt which the canine animal is experiencing. In certain embodiments, the diet is a single or varied diet.

In certain embodiments, the dietary regime is a daily, weekly, or monthly regime with a single or more than a single diet. In certain embodiments, the diet and/or dietary regime have a certain or restricted number of calories, in particular a diet and/or dietary regime which is low in calories without under nutrition (or, in other words, while maintaining nutritional requirements).

In certain embodiments, the diet and/or dietary regime and/or behavioural intervention is put in place for a length of time such that the canine animal meets the ideal weight for the breed of dog at the age, gender and sexual maturity. Such a weight is known in the art according to a body condition score on a 9 point scale (as described in Laflamme D, earlier referenced). In certain embodiments, the diet and/or dietary regime is for a period of up to about 4, up to about 6, up to about 8, up to about 16, or up to about 32 weeks. In certain embodiments, the diet and/or dietary regime is for a period of up to about a year or for longer, about two years or longer, about three years or longer, or for longer periods including for the lifetime of the animal.

In certain embodiments, the diet is a weight maintenance diet, a weight loss diet or a weight gain diet. In certain embodiments, the diet and/or the dietary regime comprises, on a dry matter basis, at least one of the following guidelines:

(i) an energy density of less than about 4100 kcal/kg;

(ii) less than about 20% w/w or less than about 15% w/w fat;

(iii) less than about 25% w/w or less than about 20% w/w carbohydrate;

(iv) more than about 30% w/w protein;

(vi) more than about 5% w/w or more than about 10% w/w total dietary fiber; and/or

(vii) combinations thereof.

In certain embodiments, the energy density is about 4100 kcal/kg, about 4000 kcal/kg, about 3900 kcal/kg, about 3800 kcal/kg, about 3700 kcal/kg, about 3600 kcal/kg, about 3500 kcal/kg, about 3000 kcal/kg, about 2500 kcal/kg, about 2000 kcal/kg, about 1500 kcal/kg, about 1000 kcal/kg or less, or any intermediate value or range thereof. In certain embodiments, the amount of fat is about 20% w/w, 19% w/w, 18% w/w, 17% w/w, 16% w/w, 15% w/w, 14% w/w, 13% w/w, 12% w/w, 11% w/w, 10% w/w, 9% w/w, 8% w/w, 7% w/w, 6% w/w, 5% w/w, 4% w/w, 3% w/w, 2% w/w, 1% w/w or less, or any intermediate value or range thereof. In certain embodiments, the amount of carbohydrate is about 25% w/w, 20% w/w, 15% w/w, 10% w/w, 5% w/w, 1% w/w or less, or any intermediate value or range thereof. In certain embodiments, the amount of protein is about 20% w/w, 25% w/w, 30% w/w, 35% w/w, 40% w/w, 45% w/w or more, or any intermediate value or range thereof. In certain embodiments, the amount of dietary fiber is about 5% w/w, 10% w/w, 15% w/w, 20% w/w, 25% w/w, 30% w/w, 35% w/w, 40% w/w, 45% w/w or more, or any intermediate value or range thereof.

Optional other components include one or more prebiotic, probiotic and/or any other ingredient. In certain embodiments, the precise diet and/or dietary regime are determined on additional factors such as the breed, age, sex, activity level, body condition and other characteristics of the canine animal.

In certain embodiments, the diet contains one or more ingredients which have a satiating effect. In certain embodiments, the diet is a weight maintenance or a weight loss diet for puppies which have a satiating effect. In certain embodiments, the maintenance diet with satiating effect can be particularly favoured as such a diet would be acceptable to reduce satiety in puppies, without reducing calories and without affecting growth. In certain embodiments, puppy weight loss/gain/management diets is tailored to a diet based around the size of the dog groups (small, medium/large, giant, as described before), or based on the dog breed or individualised per puppy.

In certain embodiments, a weight maintenance diet, a weight loss diet or a weight gain diet has a formulation selected from Table 1.

TABLE 1

| | Energy | | | | | |
|---|---|---|---|---|---|---|
| | Density | | Carbohydrate | Protein | Crude | |
| Diet | kcal/kg | Fat % | % | % | Fiber % | Calcium:Phosphorus |
| Puppy/ | 3800-4200 | 20-22 | 18-50 | 25-35 | 2-15 | 1:1.5 |
| Adolescence | | | | | | |
| Weight | | | | | | |
| Maintenance | | | | | | |
| Puppy/ | 3000-4000 | 11-20 | 25-50 | 27-38 | 4-18 | 1:1.5 |
| Adolescence | | | | | | |
| weight loss | | | | | | |
| Puppy/ | 4000-4500 | 22-26 | 15-50 | 25-40 | 2-12 | 1:1.5 |
| Adolescence | | | | | | |
| Weight Gain | | | | | | |
| Adult weight | 3400-4000 | 12-20 | 40-60 | 15-28 | 2-15 | 1:1.2 |
| Maintenance | | | | | | |
| Adult weight | 2500-3500 | 7-12 | 45-60 | 25-35 | 8-18 | 1:1.2 |
| loss | | | | | | |
| Adult Weight | 3800-4500 | 20-30 | 30-60 | 18-33 | 2-12 | 1:1.2 |
| gain | | | | | | |

In certain embodiments, the diet is an adult diet for an animal that has fully grown. In certain embodiments, the animal can be described as requiring a weight maintenance diet. In certain embodiments, the diet is a weight gain diet. A weight gain diet is often appropriate when the animal is growing, for example in puppyhood and/or adolescence. In certain embodiments, the diet is a weight loss diet. An overweight puppy, adolescent or adult animal can need a weight loss diet.

In certain embodiments, the presently disclosed subject matter also provides a diet and/or a dietary regime for use in preventing a canine animal being overweight in adulthood, wherein the diet and/or dietary regime is administered after any one of the method disclosed above is carried out. The diet and/or dietary regime can be any as herein described. In certain embodiments, the diet and/or dietary regime is for a therapeutic use. In certain embodiments, the diet and/or dietary regime is a therapeutic use for preventing obesity and/or a therapeutic use for maintaining good health.

### 4. Foodstuff

In certain non-limiting embodiments, the presently disclosed subject matter provides, as part of the diet and/or dietary regime disclosed above, any foodstuff which a canine animal can consume as part of its diet.

The foodstuff encompasses any product that a canine animal consumes in its diet. In certain embodiments, the foodstuff is a food product in its own right. In certain embodiments, the foodstuff is a dry food product, a semi-moist food product or a moist (wet) food product. In certain embodiments, the wet food product includes food that is usually sold in a container, such as a tin, pouch or tray. In certain embodiments, the wet food product has a moisture content of 70% to 90%. In certain embodiments, dry food product includes food having a similar composition but with 5% to 15% moisture, often presented as small biscuit—like kibbles. In certain embodiments, the semi-moist food product includes food having a moisture content of from above 15% up to 70%. In certain embodiments, the amount of moisture in any product can influence the type of packaging that can be used or is required. In certain embodiments, the food product, of any moisture level is ready-to-eat.

In certain embodiments, the foodstuff can be standard food products. In certain embodiments, the foodstuff is food products for companion dogs, such as food snacks (for example snack bars, cereal bars, snacks, treats, biscuits and sweet products). In certain embodiments, the foodstuff is a cooked product.

In certain embodiments, the food product incorporates meat or animal-derived material (such as beef, chicken, turkey, lamb, fish, blood plasma, marrowbone, etc. or one or more thereof). Alternatively, the foodstuff can be meat-free (preferably including a meat substitute such as soya, maize gluten or a soya product in order to provide protein). The foodstuff can contain additional protein sources such as soya protein concentrate, milk, protein, gluten, etc. In an alternative embodiment, the food product can be devoid of meat or animal derived material. In another embodiment, the food product can be gluten free or dairy free.

The foodstuff can also contain starch, such as one or more grains (e.g., wheat, corn, rice, oats, barley, etc.) or can be starch-free. The foodstuff can incorporate or be a gelatinised starch matrix. The foodstuff can incorporate one or more types of fibre such as sugar beet pulp, chicory pulp, chicory, coconut endosperm fibre, wheat fibre, etc. The foodstuff can also be newly designed products currently not available. The most suitable foodstuff can be a product as described herein which is sold as a pet food, in particular a pet food for a domestic dog. It can be convenient to provide the foodstuff in a dry format, such as dried ready-to-eat cereal products (often referred to as kibbles).

The foodstuff is preferably nutritionally complete so that the practice of the disclosed subject matter can provide a suitable nutritionally complete diet and/or dietary regime for the animal.

In certain embodiments, the foodstuff is a multi-component foodstuff. The multi-component foodstuff can comprise a dried ready-to-eat cereal product. The multi-component foodstuff can only comprise such dried ready-to-eat cereal products. Alternatively, the multi-component foodstuff can comprise a dried ready-to-eat cereal product and a wet or semi-moist product. The multi-component foodstuff can comprise individual packages of food which, when all individual packages are fed over a period of time, such as 1 day, 2 days or one week, provide the diet and/or dietary regime according to the disclosed subject matter. The individual products can be packaged as discussed below.

In certain embodiments, the foodstuff is packaged. In this way, a purchasing consumer or customer is able to identify, from the packaging, the ingredients and content of the product and confirm that it is suitable for the particular animal in question. The packaging can be metal (usually in the form of a tin or flexifoil), plastic (usually in the form of a pouch or bottle), paper or card. The amount of moisture in any product can influence the type of packaging, which can be used or is required. The foodstuff can be available as a “kit” or “pack” wherein different or the same food compositions are individually packaged and these packages are somehow joined together, for example in a box and/or with overarching packaging for the two or more packages of food compositions.

In certain embodiments, the foodstuff of the presently disclosed subject matter can be fed to an animal thrice every day, twice every day, once every day, once every two days, once every three days, once every four days, once every five days, once every six days, once a week, once every two weeks, once every three weeks, or once every month. In certain embodiments, the foodstuff can be fed to an animal one or more times per day. For example, and not by way of limitation, the foodstuff can be administered once, twice, three, four, five or more times a day. In certain embodiments, the foodstuff can be fed to an animal in a constant manner, e.g., where the animal grazes on a constantly available supply of the foodstuff.

In certain embodiments, a formulation of the foodstuff the presently disclosed subject matter can further comprise an additional active agent. Non-limiting examples of additional active agents that can be present within a formulation of the foodstuff include a nutritional agent (e.g., amino acids, proteins, fatty acids, carbohydrates, sugars, nucleic acids, nucleotides, vitamins, minerals, etc.), and/or an agent that treats or prevents overweigh and/or obesity.

In certain embodiments, the foodstuff can further contain additives known in the art. In certain embodiments, such additives are present in amounts that do not impair the purpose and effect provided by the presently disclosed subject matter. Examples of contemplated additives include, but are not limited to, substances that are functionally beneficial to treat or prevent overweigh and/or obesity, substances with a stabilizing effect, organoleptic substances, processing aids, substances that enhance palatability, coloring substances, and substances that provide nutritional benefits. In certain embodiments, the stabilizing substances include, but are not limited to, substances that tend to increase the shelf life of the composition. In certain embodiments, such substances include, but are not limited to, preservatives, synergists and sequestrants, packaging gases, stabilizers, emulsifiers, thickeners, gelling agents, and humectants. In certain embodiments, the emulsifiers and/or thickening agents include, for example, gelatin, cellulose ethers, starch, starch esters, starch ethers, and modified starches.

In certain embodiments, the additives for coloring, palatability, and nutritional purposes include, for example, colorants; iron oxide, sodium chloride, potassium citrate, potassium chloride, and other edible salts; vitamins; minerals; and flavoring. The amount of such additives in a composition typically is up to about 5% (dry basis of the composition).

In certain embodiments, the foodstuff is a dietary supplement. In certain embodiments, the dietary supplements include, for example, a feed used with another feed to improve the nutritive balance or performance of the total. In certain embodiments, the supplements include compositions that are fed undiluted as a supplement to other feeds, offered free choice with other parts of an animal's ration that are separately available, or diluted and mixed with an animal's regular feed to produce a complete feed. The AAFCO, for example, provides a discussion relating to supplements in the American Feed Control Officials, Inc. Official Publication, p. 220 (2003). Supplements can be in various forms including, for example, powders, liquids, syrups, pills, encapsulated compositions, etc.

In certain embodiments, the foodstuff is a pet food. In certain embodiments, the pet food composition is a treat. In certain embodiments, treats include, for example, compositions that are given to an animal to entice the animal to eat during a non-meal time. In certain embodiments, the pet food composition is a treats for canines include, for example, dog bones. Treats can be nutritional, wherein the composition comprises one or more nutrients, and can, for example, have a composition as described above for food. Non-nutritional treats encompass any other treats that are non-toxic.

### 5. Device and System

In certain non-limiting embodiments, the presently disclosed subject matter also provides a device, a system and an application for the method disclosed in the instant application, of determining whether a canine animal is predisposed to being overweight in adulthood, or of preventing a canine animal from being overweight in adulthood. The device, system and/or application enable a user, such as a caretaker or owner to evaluate the weight gain and take action by themselves, or with the aid of a healthcare professional/veterinarian to ensure a healthy bodyweight of a canine animal is achieved and/or maintained.

In certain embodiments, a device is used to carry out the methods disclosed in the instant application. In certain embodiments, the device is configured to accept a user input. In certain embodiments, the user input comprises the first weight of the animal at birth according to step (i) of the method disclosed in the instant application, of determining whether a canine animal is predisposed to being overweight in adulthood, or of preventing a canine animal from being overweight in adulthood. In certain embodiments, a further user input is configured to be accepted, the further user input comprising the second weight of the animal according to step (ii) of the method. In certain embodiments, the device automatically (or on request) performs an analysis of step (iii) of the method, i.e., determining the weight gain between the input in steps (i) and (ii) of the method of the disclosed subject matter. The analysis provides the weight gain between the dates of steps (i) and (ii) and provides output information. In certain embodiments, the device provides a message with the output of step (iii). In certain embodiments, the message comprises a warning, wherein the weight gain is 125% or more as set out according to the method of the disclosed subject matter. In one embodiment, the results of the method are provided by the device in a user interface. In certain embodiments, the device provides a recommendation of nutritional suggestions according to a diet and/or a dietary regime disclosed in the instant application.

In certain embodiments, the device may be specially constructed for the required purposes, or it may comprise a general purpose computer selectively activated or reconfigured by a computer program/application stored in the computer. Such a computer program/application may be stored in a computer readable storage medium, such as, but is not limited to, read-only memories (ROMs), random access memories (RAMs), EPROMs, EEPROMs, flash memory, magnetic or optical cards, any type of disk including floppy disks, optical disks, CD-ROMs, and magnetic-optical disks, or any type of media suitable for storing electronic instructions, and each coupled to a computer system interconnect.

In certain embodiment, the device comprises a processor that executes an application that directs the device to provide data fields for entry of user input relating to step (i) and step (ii). In certain embodiment, the application uses the processor to evaluate the risk of the animal being predisposed to overweight in adulthood. In certain embodiments, the application is an easily navigable application, e.g., online, to carry out a method disclosed in the instant application.

In certain embodiment, the device is a tablet, smartphone, desktop computer, laptop computer or personal digital assistant. In certain embodiment, the device is a mobile device, such as a smartphone and a tablet.

In certain embodiments, a system is also provided for the method disclosed in the instant application, of determining whether a canine animal is predisposed to being overweight in adulthood. In certain embodiments, the system comprises a database connected to a remotely located device disclosed herein. In certain embodiments, the device comprises a processor executing an analysis that evaluates a determination according to the method disclosed in the instant application

Unless specifically stated otherwise as apparent from the following discussion, it is appreciated that throughout the description, discussions utilizing terms such as “processing” or “computing” or “calculating” or “determining” or “displaying” or “analyzing” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission, or display devices.

The algorithms and displays presented herein are not inherently related to any particular computer or other device. Various general purpose systems may be used with the application in accordance with the teachings herein, or it may prove convenient to construct a more specialized device to perform the required method operations. The structure for a variety of these systems will appear from the description above. In addition, the present embodiments are not described with reference to any particular programming language, and various examples may thus be implemented using a variety of programming languages.

All preferred features and/or embodiments of the methods and the diets/dietary regimes disclosed in the instant application apply to the device, the system and the application.

### EXAMPLES

The presently disclosed subject matter will be better understood by reference to the following Examples, which are provided as exemplary of the disclosed subject matter, and not by way of limitation.

### Example 1

In order to identify factors which can help explaining development of adult overweight status in dogs, a longitudinal study of 2 years was conducted on 24 female Beagle dogs of the same age, sexual status and raised under identical environmental conditions. To analyze the data, three groups of dogs were constituted: i) ideal weight (IW, n=9), ii) slightly overweight (OW1, n=6) and iii) severely overweight or obese (OW2, n=9), according to a PCA at 2 years of age, on the values of fat-free mass, % fat mass and pelvic circumference. The relevant predictive factors of a marked overweight status in adult dogs were statistically selected among the following factors: parental characteristics, growth pattern, energy balance and plasma factors. The animals were measured using standard weighing scales and the birth weights were measured within 24 hours of birth. Logistic regression analysis highlighted that the percentage of weight gain during the 2 first weeks of life, i.e., the neonatal growth rate, was higher in the OW2 group than in the IW (p=0.015) and the OW1 groups (p=0.040). Seventy percent of the dogs displaying a neonatal growth rate greater than 125% belonged to the OW2 group. In conclusion, the present study established that severely overweight adult dogs had a higher neonatal growth rate.

### Introduction

Overweight and obese dogs carry excessive accumulation of adipose tissue which is translated to a body weight (BW) >15% and >30% above ideal BW, respectively. The main reason for excess weight gain in healthy dogs is a positive imbalance between energy intake and energy expenditure. Some obesity-related outcomes are reversible by restricted energy intake and by increased activity through a weight loss program. However, only half of the dogs entering such program reach their target body weight and among them, only half of them succeed in maintaining their optimal body weight over the long term. Among the plasma factors related to energy intake, some of them were reported to differ between obese dogs and normal weight dogs, such as insulin, ghrelin, leptin and adiponectin. Obesity in dogs also results in low-grade systemic inflammation which can contribute to the genesis of metabolic disorders.

Numerous investigations have identified risk factors which were associated with excess weight, in overweight or obese but otherwise healthy dogs in various countries. The main reported risk factors are neutering especially in females, high feeding frequency, sedentary lifestyle and specific breeds or cross-breeds.

The aims of this study were (i) to show that spontaneous differences in weight gain could occur among dogs raised in the same conditions (of age, sex, sexual status, breed, diet and environment) and (ii) to identify early predictive factors such as parental and neonatal characteristics, energy intake or expenditure, or plasma biomarkers, associated with overweight status in adulthood.

A listing of abbreviations used are set forth below:

BCS Body condition score

BW Body weight

BW_{b} Body weight at birth

FM % Percentage fat mass

FFM Fat free mass

GR_{2W} Growth rate in the 2 first weeks of life

PC Pelvic circumference

### Methods

Animals.

Twenty-four female Beagle dogs were studied from birth to 24 months of age. They were born from 10 different mothers and 7 different fathers, housed in the same breeding centre (ISOQUIMEN, Barcelona, Spain), weaned at 10 weeks, and neutered at the age of 8 months. All dogs received an annual veterinary check-up, were vaccinated and received worming treatments every 3 months.

Housing.

Throughout the study, the dogs lived in the same environment and were fed in the same manner. Prior to weaning, puppies and mothers were housed by litter in the breeding centre of Isoquimen. After weaning, dogs were relocated to Oniris (Nantes, France) and were housed in pairs in closed outdoor enclosures of 4 m². During feeding time, half the dogs were fed in their enclosures, while the remaining dogs were housed in two 31 m² outdoor enclosures. The outdoor enclosures disposed of a sheltered place to sleep.

Dogs were taken for a walk at least 15 min twice a week and had access to 1 h/day of free time in a closed garden of 400 m² enriched with agility equipment.

Diet.

All diets were the same diet (protein: 30%, fat: 22%, 4012 kcal/kg) and it was offered ad libitum to both mothers and puppies from conception to weaning.

Data Analysis.

All statistical analyses were performed using the R software. Graphs were prepared using GraphPad Prism software. Significant differences have been considered for a risk of first type error of 0.05.

As a principal component analysis (PCA) has to be performed on quantitative variables, it was conducted on the three following variables: pelvic circumference (PC), fat free mass (FFM) and proportion of fat mass (FM %) at the age of 24 months (FactoMineR package, in R software). Consequently, BCS, which is a discrete variable, was added to the PCA as a supplementary variable as well as the parental and gestational variables.

A hierarchical classification on principal components was conducted on the previous variables to allot the dogs into three distinct groups.

Logistic regression analyses were performed on the 3 groups by pairs (IW-OW1; OW1-OW2, IW-OW2) to discriminate the groups by

i) Parental and gestational variables, and

ii) BCS, FM % or FFM before 2 years old

An estimation of an accurate threshold was processed after the logistic regression analysis, on the identified discriminant factors. The goodness of fit was explored through an analysis of deviance table+.

### Results

All dogs remained healthy during the study and their serum biochemistry values remained within normal ranges.

Constitution of 3 Groups Based on the Dogs' Biometric Data at 24 Months of Age

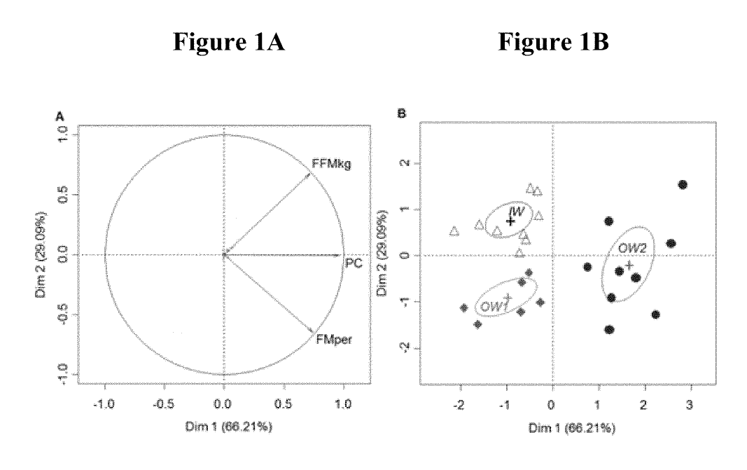

In order to categorize dogs according to its overweight status, a PCA was performed on PC (cm), FM % (%) and FFM (kg) at 24 months of age (FIG. 1). The two first axes explained 95.3% of the total variation. A hierarchical clustering on principal components revealed 3 well separated groups, which could be described as: dogs with ideal (optimal) weight (IW, n=9), slightly overweight dogs (OW1, n=6) and severely overweight or obese dogs (OW2, n=9).

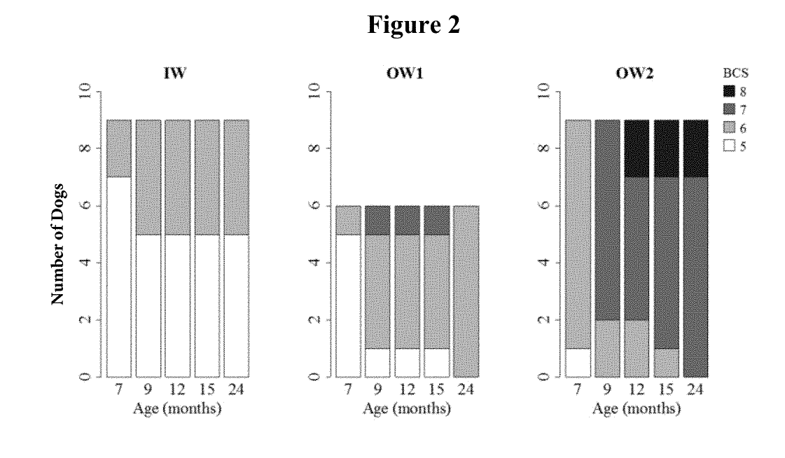

At 24 months, the BW, FFM, FM % and PC values were significantly higher in the OW2 group than those in the OW1 group (p<0.001, p<0.001, p=0.009, p<0.001, respectively). The BW, FM % and PC were significantly higher in the OW2 group than in the IW groups (all p<0.001). FFM was significantly lower in the OW1 group than in the IW group (p=0.010), whereas FM % was significantly greater in the OW1 compared to the IW group (p<0.001). The distribution of BCS in the groups at 24 months was in line with the groups' characteristics (FIG. 2). Among the groups, 6 dogs in the IW group had a BCS of 5/9, all dogs in the OW1 group displayed a BCS of 6/9 and all dogs of the OW2 group had a BCS superior to 6/9. The withers height was significantly higher in the OW2 (42 (1.5) cm) group than those in the IW (40.2 (1.4) cm; p=0.037) and the OW1 groups (39.0 (2.5) cm; p=0.003). The withers height was not significantly different between the IW and the OW1 groups (p=0.201).

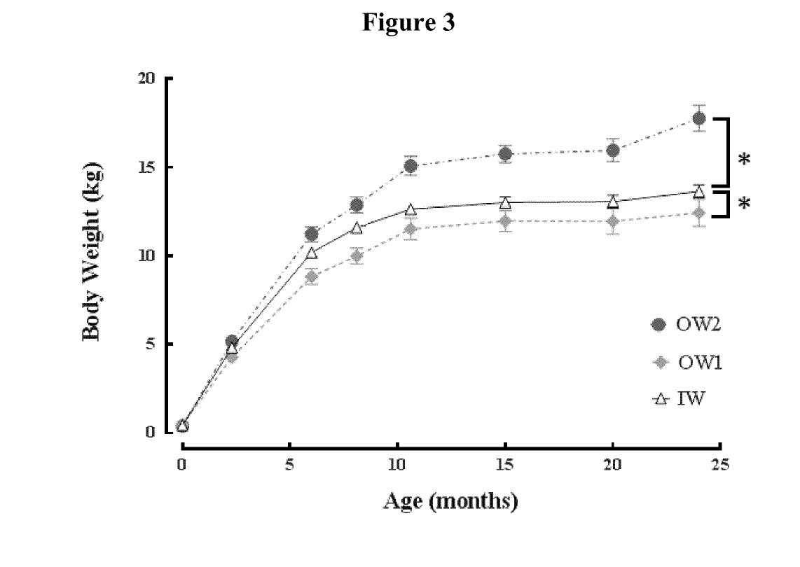

Excess weight above optimal weight was estimated for each group as described in the methods. FIG. 3 provides body weight (kg) over age (months) of the 24 dogs, plotted between the three groups designated as OW2, OW1, and IW. The percentage of excess weight of each group was found to be significantly higher in the OW2 group than in OW1 (p=0.002) and the IW groups (p<0.001), and higher in the OW1 group than in the IW group (p=0.001).

Physiological Periods of Weight Gain—Birth to Weaning

The BW_{b} (IW: 0.44 (0.7) kg; OW1: 0.47 (0.14) kg; OW2: 0.39 (0.06) kg) did not differ among the groups.

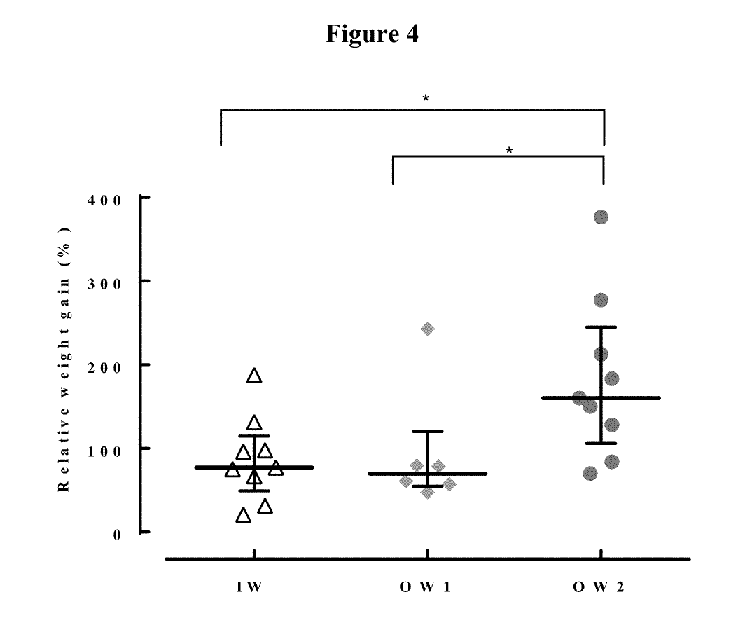

The growth rate during the first two weeks of life (GR_{2W})(IW: 87.1 (50.5) %; OW1: 94.4 (73.8) %; OW2: 182.4 (30.2) %) was significantly higher in the OW2 than in the IW (p=0.015) and the OW1 groups (p=0.040) (FIG. 4). A logistic regression analysis highlighted that GR_{2W} discriminated the OW2 from the IW (p=0.051) groups. Moreover, a GR_{2W} of 125% during the first two weeks of life was detected as an accurate threshold to predict OW2 status in adulthood. Hence, among the 10 dogs which displayed a GR_{2W} higher than 125%, 7 belonged to the OW2 groups in adulthood (2 to IW and 1 to OW1). Furthermore, GR_{2W} was significantly correlated with FM % (data not shown, p=0.038) independently of groups.

### Discussion

This is a first longitudinal study in growing dogs that aimed to identify predictive factors which could explain overweight status in adulthood. The first aim of this study was to determine whether spontaneous differences of weight gain could occur in a homogenous population of dogs fed ad libitum. The population was composed of female Beagle dogs of the same age, sterilised and raised in identical conditions from conception to 2 years of age. The Beagle breed was chosen because its propensity to develop obesity, in addition to other risks such as age, sex, sexual status and environmental conditions.

Dogs were allotted, a posteriori, into 3 groups according to FM %, FFM and PC at 24 months of age. The dogs that spontaneously became severely overweight or obese (BCS of 7 or 8) accounted for 37.5% of the entire population (OW2), whereas the dogs with an optimal weight (OW1, BCS of 5 or 6), also accounted for 37.5% of the population. The proportion of overweight dogs (62.5%) is in line with the reported prevalence of overweight dogs worldwide. Given that the dogs were fed ad libitum, it is worthy to note a subset of the population did not become overweight. These results confirm that some dogs could be more susceptible to gain body fat than others of the same breed and housed under the same environmental conditions.

The second aim of this study was to identify, as early as possible, predictive markers of overweight status or excess weight gain in adulthood. In contrast to human and other animal studies, body weight at birth was neither correlated to the overweight status, fat-free mass at adulthood nor correlated to parental body weight. Given that the normal weight for neonatal puppies at 10 days of age is twice that at birth, the growth rate in the first 2 weeks of life (GR_{2W}) was assessed. As 78% percent of dogs which had a GR_{2w} greater than 125% belonged to the OW2 group, GR_{2w} was identified as the earliest predictive factor of the adult overweight status in the present study.

The neonatal finding can be used by breeders to identify the dogs which should be dietary restricted. The adolescence findings might be used by veterinarians to assess the risk of becoming overweight in adulthood and to deliver specific nutritional advice.

Although the presently disclosed subject matter and its advantages have been described in detail, it should be understood that various changes, substitutions and alterations can be made herein without departing from the spirit and scope of the disclosed subject matter as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A method of determining whether a canine animal is predisposed to being overweight in adulthood, the method comprising:
determining a first weight of the canine animal at birth;
(ii) determining a second weight of the canine animal at a date between about 13 to about 15 days of age; and
(iii) determining a weight gain between the dates of steps (i) and (ii),
wherein a weight gain of about 125% or more between the dates of steps (i) and (ii) determines that the canine animal is predisposed to being overweight in adulthood.

2. The method of claim 1, wherein the date of the second weight is about 14 days of age.

3. The method of claim 1, wherein adulthood is from about 2 years of age onward.

4. The method of claim 1, wherein overweight is obese.

5. The method of claim 1, wherein the canine animal is a domestic dog.

6. The method of claim 1, wherein the canine animal is determined to be predisposed to be overweight in adulthood, and a diet or a dietary regime is administered to maintain or achieve a healthy bodyweight.

7. A diet or a dietary regime for a canine animal, which has been determined as predisposed to being overweight in adulthood according to the method of claim 1.

8. The diet or a dietary regime as claimed in claim 7, wherein the dietary regime contains, on a dry matter basis,
(i) an energy density of less than about 4100 kcal/kg;
(ii) less than about 20% w/w or less than about 15% w/w fat;
(iii) less than about 25% w/w or less than about 20% w/w carbohydrate;
(iv) more than about 30% w/w protein; or
(v) more than about 5% w/w or more than about 10% w/w total dietary fiber.

9. A method of preventing a canine animal from being overweight in adulthood comprising:
(i) determining a first weight of the canine animal at birth;
(ii) determining a second weight of the canine animal at a date between about 13 to about 15 days of age; and
(iii) determining a weight gain between the dates of steps (i) and (ii), wherein a weight gain of 125% or more between the dates of steps (i) and (ii) determines that the canine animal is predisposed to being overweight in adulthood, and
(iv) administering a maintenance diet for a maintenance period, or administering a weight adjustment diet for a weight adjustment period.

10. The method of claim 9, wherein the weight adjustment diet is a weight loss diet or a weight gain diet.

11. The method of claim 9, further comprising determining a maintenance body weight of the canine animal, comparing the maintenance body weight to a healthy body weight to calculate a body weight deviation, and administering a weight adjustment diet if the body weight deviation is about 5% or greater.

12. The method of claim 11, wherein the weight adjustment diet is a weight loss diet if the body weight deviation is above the healthy body weight.

13. The method of claim 11, wherein the weight adjustment diet is a weight gain diet if the body weight deviation is below the healthy body weight.

14. The method of claim 9, wherein administering a maintenance diet or administering a weight adjustment diet is replaced or supplemented by administering a therapeutic agent.

15. The method of claim 14, wherein the therapeutic agent is capable of treating or preventing obesity.

16. A diet or a dietary regime for use in preventing a canine animal from being overweight in adulthood, wherein the diet or the dietary regime is administered after the method of claim 1 is carried out, wherein the canine animal has been determined as predisposed to being overweight in adulthood.
